(19) United States
(12) Patent Application Publication
(10) Pub. No.: US 2005/0037080 A1
(43) Pub. Date: Feb. 17, 2005

(54) Air treatment gel and method for its preparation

(76) Inventors: Maurice Gerard Lynch, Waterloo (BE); Ana Chordi Miranda, Etterbeek (BE)

Correspondence Address: Patent Administrator;FMC Corporation, 1735 Market Street, Philadelphia, PA, 19103, US

(21) Appl. No.: 10/422,449

(22) Filed: Apr. 24, 2003

Related U.S. Application Data
(60) Provisional application No. 60/375,590, filed on Apr. 25, 2002.

(51) Int. Cl. A61K 9/14

(52) U.S. Cl. 424/488

(57) ABSTRACT

A gel useful as an air freshener, and methods for its preparation, are disclosed. The gel contains an active substance, such as a fragrance oil; a gel-forming polymer, such as alginate and/or a pectic substance; a divalent cation or mixture of divalent cations, preferably calcium ion, which gels the gel-forming polymer; at least one added polymer that is not gelled by the divalent cation or mixture of divalent cations, such as carboxymethyl cellulose; and water. Gelation does not require heating of the composition before gelation and does not begin until a pH modifier, such as gluconic acid δ-lactone, is added. Thus, the manufacturing method has lower energy costs and improved production efficiencies.

This application claims the benefit of U.S. Provisional Application No. 60/375,590, filed Apr. 25, 2002.

### FIELD OF THE INVENTION

This invention relates to air treatment gels. In particular, this invention relates to the preparation of air treatment gels comprising a gel-forming polymer that is gelled with a gelling agent comprising a divalent cation.

### BACKGROUND OF THE INVENTION

Air treatment gels continuously release volatile air treatment components from the gel when exposed to the atmosphere. The volatile air treatment components can include air freshening ingredients, such as perfumes, which provide a pleasant odor and/or reduce or mask unpleasant odors, and/or ingredients such as disinfectants, bactericides, and insecticidal materials. The air treatment components typically are compatible with each other and the other components of the gel, are dispersible in water, and volatilize slowly at ambient temperature.

An air treatment gel typically comprises an aqueous medium containing the volatile air treatment components, and at least one gel-forming polymer, which gels the largely aqueous medium. The air treatment component, gel-forming agent and water typically comprise over 80 wt %, i.e. about 85 to 99 wt %, more typically over 90 wt %, i.e. about 96 to 99 wt %, of the aqueous medium, of which about 1 to 10 wt % is the volatile air treating components, and about 1 to 4 wt % gel-forming polymer. Numerous gel-forming polymers have been described. See, for example, Turner, U.S. Pat. No. 2,691,615; Modi, U.S. Pat. No. 5,741,482; Steer, U.S. Pat. No. 4,755,377; and Sakurai, U.S. Pat. No. 4,137,196. However, many of these gel compositions have serious disadvantages, for example: (1) syneresis, i.e., the spontaneous separation of the aqueous medium from the gel due to contraction of the gel during storage; (2) expense, i.e. they use large amounts of expensive gel-forming polymers, which can make them uneconomical; and (3) high energy consumption during manufacturing. Thus, a need exists for an air treatment gel that has very low syneresis yet is economical to manufacture.

### SUMMARY OF THE INVENTION

In one aspect the invention is a gel. The gel comprises:
(a) an active substance,
(b) a gel-forming polymer containing gelling sites, the gel-forming polymer selected from the group consisting of alginate, pectic substances, and combinations thereof, and
(c) at least one added polymer;
   in which:
the gel-forming polymer is gelled;
the gelling sites of the gel-forming polymer are less that 100% saturated with a divalent cation or mixture of divalent cations; and
the at least one added polymer is not gelled by the divalent cation or mixture of divalent cations.

This gel has very low syneresis yet is economical to manufacture. Unlike commonly used gels, this gel can be manufactured without heating, resulting in reduced energy costs of production and lower capital investment in production equipment.

In another aspect, the invention is a method for preparing the gel, the method comprising combining:
(a) an active substance,
(b) at least one gel-forming polymer containing gelling sites, the gel-forming polymer selected from the group consisting of alginate, pectic substances, and combinations thereof,
(c) at least one gelling agent comprising a divalent cation capable of gelling the gel-forming polymer,
(d) at least one added polymer,
(e) a pH modifier; and
(f) water;
   in which:
the divalent cation is present in a molar amount less than that required to completely saturate the gelling sites of the gel-forming polymer, and
the added polymer is not gelled by the divalent cation.

Preferably, at least a portion of the divalent cation is solubilized, i.e. becomes available to react with the gelling sites, responsive to a decrease in pH in the gel-forming composition. A preferred gel-forming polymer is alginate having a G content of about 40% to about 90%. A preferred added polymer is carboxymethyl cellulose, preferably carboxymethyl cellulose having a Brookfield viscosity in a 1% aqueous solution at 25° C. of between 10,000-20,000 cps. A preferred divalent cation is the calcium (+2) ion. A preferred gelling agent is dicalcium phosphate dihydrate. A preferred pH modifier is glucono delta lactone. In yet another aspect, the invention is a gel prepared by this method.

### DETAILED DESCRIPTION OF THE INVENTION

In the specification, examples, and claims, unless otherwise indicated, percents are percents by weight. Except where indicated by context, terms such as “gel-forming polymer,” “surfactant,” “added polymer,” “gelling agent,” “pH modifier,” “divalent cation,” and similar terms also refer to mixtures of such materials.

In one aspect, the invention is a gelled composition, or gel, useful as an air freshener, formed by gelation of a gel-forming composition. The gel comprises a gelled gel-forming polymer, an added polymer, and an active substance. Other ingredients that are conventional components of gels, such as preservatives, colorants, and surfactants, for example, may also be present. The balance of the gel is water, bound to and/or immobilized by either the gelled polymer and/or to the added polymer.

### Gel-Forming Polymer

The gel-forming polymer is a polyuronide such as an alginate, pectic substances or a combination thereof. Alginate is a water soluble salt of alginic acid. Alginic acid is a polyuronic acid made up of two uronic acids: D-mannuronic acid and L-guluronic acid. The ratio of mannuronic acid and guluronic acid varies with factors such as seaweed species, plant age, and part of the seaweed (e.g., stem, leaf). Alginic acid in the form of mixed water insoluble salts, in which the principal cation is calcium, is found in the fronds and stems of seaweeds of the class Phaeophyceae, examples of which are Fucus vesiculosus, Fucus spiralis, Ascophyllum nodosum, Macrocystis pyrifera, Alaria esculenta, Eclonia maxima, Lessonia nigrescens, Lessonia trabeculata, Laminaria japonica, Durvillea antarctica, Laminaria hyperborea, Laminaria longicruris, Laminaria digitata, Laminaria saccharina, Laminaria cloustoni, and Saragassum sp.

Methods for the recovery of water-insoluble alginic acid and its water-soluble salts, especially sodium alginate, are well known. They are described, for example, in Green, U.S. Pat. No. 2,036,934, and Le Gloahec, U.S. Pat. No. 2,128,551.

Alginic acid is substantially insoluble in water. It forms water-soluble salts with alkali metals, magnesium, ammonium, lower amines, and certain other organic bases. These salts form viscous aqueous solutions. The salts are soluble in aqueous media above pH 4, but are converted to alginic acid when the pH is lowered below about pH 4. Water-insoluble calcium alginate is formed if calcium ion is present in the medium.

Pectic substances include, e.g., pectins, pectate, etc. and are a naturally occurring polysaccharide found in the roots, stems, leaves, and fruits of various plants, especially the peel of citrus fruits such as limes, lemons, grapefruits, and oranges. They contain polymeric units derived from D-galacturonic acid. About 20-60% of the units derived from D-galacturonic acid, depending on the source of the pectin, are esterified with methyl groups. These are commercially known as high methoxy pectin and low methoxy pectin, the latter also including amidated pectins. Pectate (pectinate) is fully de-esterified pectin with up to 20% of the units derived from D-galacturonic acid.

Gels are formed by the immobilization of water within a network created by ionic crosslinking of the gelling sites of the polymer chains of the gel-forming polymer. The alginates and pectic substances all contain gelling sites that can react with the divalent cation. In the case of alginates these gelling sites are L-guluronic acid units occurring in series (G blocks) on the polymer chain. In the case of pectic substances, the gelling sites are D-galacturonic acid units occurring in series on the polymer chain. Each ionic crosslink occurs as the result of an ionic reaction between one divalent cation and two gelling sites, each of which is located on different polymer chains. Ionic reaction to link polymer chains is not the same as, and should not be confused with, crosslinking in which a chemical bond, for example, a carbon-carbon bond or a carbon sulfur bond, is formed between two polymer chains.

The gel of the invention is considered to be mechanically homogeneous. Mechanically homogenous means that the reacted gelling sites of the alginate, pectin or combination thereof are evenly distributed throughout the gel as produced by the process of this invention. This consistency in gel structure gives the product a greater stability on storage as compared to those that are not mechanically homogenous. This can be determined by measuring the syneresis on storage at 20° C. of less than 1% at 18 hours in a sealed container and less than 2% syneresis at 20° C. after one month in a sealed container. Because end use applications have different storage stability criteria, the optimized formulations to provide acceptable low syneresis for heat storage stability or thermal cycle may not produce as low a syneresis value at room temperature even though these gels are mechanically homogenous.

### Gelling Agent

The gelling agent comprises a divalent cation or mixture of divalent cations capable of gelling the gel-forming polymer. Suitable divalent cations include, for example, barium, strontium, divalent iron, calcium, and zinc cations. Preferred divalent cations are divalent metal cations, more preferably the calcium (+2) cation.

Any salt or combination of salts that provides the desired gelling divalent cation or mixture of divalent cations can be used as the gelling agent, so long as the resulting composition remains pumpable and is not capable of forming a gel until addition of the pH modifier. Salts that provide the calcium ion are at least slightly water soluble at neutral pH, preferably with increased solubility of divalent cation at acidic pH, and include, for example the following calcium salts, their hydrates, and mixtures thereof: calcium carbonate, dicalcium phosphate, tricalcium phosphate, and tricalcium citrate. A preferred gelling agent is dicalcium phosphate dihydrate (CaHPO₄.2H₂O; CAS No. 7789-77-7). Dicalcium phosphate dihydrate, not only provides the divalent cation necessary for gel formation, it also provides a phosphate buffer.

An important aspect of this invention is that the gelling agent is present in an amount such that the resulting gel contains gelling sites that are not reacted with divalent cations; i.e., the divalent cation or mixture of divalent cations is present in a molar amount less than that required to saturate 100% of the gelling sites of the gel-forming polymer. Divalent cations available from any source, including, for example, from the water or any other components used to from the gel-forming composition, must be included in the calculation of % saturation. Divalent cations that either are not available (e.g. due to insolubility) or that are not capable of ionically crosslinking the gelling sites must be excluded from the calculation of % saturation.

### Added Polymer

The added polymer is a polymer that binds or immobilizes water and is not gelled by the divalent cation. The added polymer reduces syneresis of the gel on storage. Because air treatment gels, especially air treatment gels used in automotive applications, may be subjected to a wide range of temperatures, both prior to and during use, it is advantageous to include an added polymer that reduces syneresis both at high temperatures and during the freeze/thaw cycles.

Typical added polymers are carboxymethyl cellulose (CMC), cellulose esters, and xanthan. Cellulose esters include the well-known esters of cellulose, for example hydroxyethyl cellulose, hydroxypropyl cellulose, hydroxypropyl methyl cellulose, cellulose acetate, and cellulose acetate butyrate. Xanthan is a commercially available high molecular weight polysaccharide having a molecular weight of about 1,000,000 to about 10,000,000.

A preferred added polymer is carboxymethyl cellulose. Preferably, the carboxymethyl cellulose is a high viscosity carboxymethyl cellulose. High viscosity carboxymethyl cellulose has a Brookfield viscosity of a 1% aqueous solution at 25° C. of at least about 5,000 mPa s (cps), measured with a Brookfield viscometer using spindle 4 at 30 rpm. High viscosity carboxymethyl cellulose is well known in the art and is sold as, for example, BLANOSE® CMC-7H9 and BLANOSE® CMC-9H4 (Hercules, AQUALON® Division, Wilmington, Del., USA). Preferably a 1% solution of the carboxymethyl cellulose in water has a Brookfield viscosity of between about 10,000 cP (mPa s) and about 20,000 cP (mPa s) at 25° C., measured with a Brookfield viscometer using spindle 4 at 30 rpm.

### pH Modifier

The pH modifier is added during preparation of the gel composition to lower the pH. As the pH of the composition is lowered, divalent cation, typically a divalent metal cation such as the calcium (+2) ion, becomes available from the gelling agent. The divalent cation reacts with the gelling sites of the gel-forming polymer, and gelation takes place.

An important aspect of this invention is that the pH is slowly lowered during gel formation. Thus, a pH modifier that slowly lowers the pH is preferred. Acids that provide a buffering action and/or materials that slowly generate acid, such as anhydrides, esters, amides, lactones, and lactams, which slowly generate acids by chemical reaction, can be used as the pH modifier. Various materials, especially those that slowly generate an organic acid that buffers the gel-forming composition, may be used as the pH modifier. These include, for example, lactic acid lactone, glycolic acid lactone, and glucono delta lactone. Combinations of materials, in which one slowly generates acid and the other provides a buffering effect, may be used.

A preferred pH modifier is glucono delta lactone (gluconic acid δ-lactone), but other pH modifiers that slowly lower the pH could also be used. Glucono delta lactone (GDL) provides optimum results in terms of gel strength and storage stability because it slowly reduces the pH, allowing gelation to occur in a very controlled manner, which aids formation of a mechanically homogeneous gel. After 20 minutes, the gel is strong enough to withstand packaging and transport. After 12 hours the gel achieves its maximum strength.

### Active Substance

The gel typically comprises an active substance. The active substance typically comprises a perfume constituent, i.e., a compound or mixture of compounds capable of imparting a pleasing fragrance. Other ingredients, such as disinfectants, bactericides, fungicides, deodorants, pest repellants, insecticides, and mixtures thereof, may be present in addition to, or in place of, the perfume constituent. Preferably these components are volatile materials at room temperature, compatible with the other ingredients in the gel, and dispersible in an aqueous medium.

The perfume constituent typically comprises one or more essential oils obtained from leaves, flowers, fruits, roots and wood; from animal sources, and from resinous extracts. Typically perfume constituents include, for example, essential oils, which are distillable, odoriferous, oily products obtained from the leaves, stems or flowers of numerous plants and usually carry the savory or odorous principles of the plant. Typical essential oils are oil of rose, oil of lime, oil of lemon, oil of lemon grass, oil of camphor, oil of cinnamon, oil of spearmint, oil of peppermint, oil of wintergreen, oil of cedar wood, oil of pine, oil of eucalyptus, and oil of fir Canadian. These essential oils may also be used in combination with fragrances such as aromatic esters, aldehydes, ketones, and other compounds known to those skilled in the art of blending fragrances. Synthetically-derived fragrancing materials, including synthetically-prepared active ingredients of the essential oils, may also be used as the perfume constituent, typically in admixture or in admixture with the natural substances. Synthetic materials include, for example, menthol, camphor, and methyl salicylate. The perfume constituent is frequently a complex mixture of one or more essential oils and/or synthetic fragrancing materials, and is typically available from a perfume supplier. The perfume constituent is often provided in a suitable vehicle, such as an alcohol or other solvent.

In lieu of or in addition to the perfume constituent, the active substance may comprise one or more other volatile air treating constituents known to the art, for example, pheromones, bactericides, insect attractants and repellants, animal attractants and repellants, insecticides, fungicides, and pharmaceutical and veterinary drugs.

A conventional surfactant is typically used to disperse the active substance in the aqueous medium used to form the gel. The surfactant should have little or no odor. Typical surfactants include, for example, non-ionic surfactants, such as ethoxylates, sorbitan esters and polyethoxylates of sorbitan esters. Typical ethoxylates include ethoxylated alkyl phenols, such as ethoxylated octyl phenol and ethoxylated nonyl phenol; ethoxylated alcohols, such as ethoxylated lauryl alcohol, ethoxylated cetyl alcohol, and ethoxylated glycerin; glycerol mono/dilaurate polyglycol ether; ethoxylated castor oil; and ethoxylated hydrogenated castor oil.

### Other Ingredients

The gel may comprise a preservative or mixture of preservatives, i.e., bactericides or fungicides to inhibit microbial or fungal growth in the air treatment gel. Typically, the gel comprises about 0.001% to about 2%, preferably from about 0.001% to about 0.5% of the preservative or mixture of preservatives. Any of the common bactericides or fungicides known in the art may be used, such as 2-chloroacetamide, sodium benzoate, and methyl-, butyl- or propyl-p-hydroxybenzoate.

The gel may comprise a colorant, typically a dye or mixture of dyes, which imparts color to the gel. When present, colorant typically comprises about 0.05% or less of the gel.

### Water

The balance of the composition is water, immobilized by either the gel-forming polymer and/or the added polymer. Although distilled or deionized water may be used to prepare the compositions, tap water may also be used. However, if tap water is used, the amount of gel-forming divalent cations, especially the amount of calcium ion, present in water must be considered in calculating the saturation level of the gel-forming polymer.

### Composition

The gel-forming composition preferably comprises: gel-forming polymer, 0.5% to 2.0%, preferably 0.8% to 1.5%; added polymer, 0.25 to 3%, preferably 0.3% to 1.5%; and pH modifier, 0.2 to 1.0%. The amount of gelling agent depends on the type and amount of gel-forming polymer. The amount of gel-forming divalent cation present is less than the amount required to completely saturate the gelling sites of the gel-forming polymer. Suitable levels of the active substance range from 0.2 to 10%, preferably from about 0.5 to about 10%, by weight of the composition. The amount of surfactant depends on the nature of the surfactant and the fragrance oil, but is typically about 0.5% to about 15%, more typically 1% to about 10%.

### Gel Preparation

As described above, an important aspect of this invention is that the gelling sites of the gel-forming polymer are less than 100% saturated with the divalent cation. An excess of divalent cations will over-saturate the gel-forming polymer, producing deleterious effects in the gel.

As used herein, “less than 100% saturated with the divalent cation” means that the divalent cation is present in a molar amount less than that required to completely (100%) saturate the gelling sites of the gel-forming polymer. For example, when sufficient divalent cations are present to react with all available gelling sites (L-guluronic acid units in the case of alginate), without any excess divalent cation remaining, the gel-forming polymer is considered to be saturated. The amount of divalent cation required to completely saturate the gelling sites of the gel-forming polymer is considered to be 1 mM divalent cation per 2 mM L-guluronic acid in the case of alginates and 1 mM divalent cation per 2 mM D-galacturonic in the case of pectic substances. Thus, any amount less than these is considered to be an amount less than that required to completely saturate the gelling sites of gel-forming polymer.

In alginate, the strength of gels formed by reaction of alginate with divalent cations is known to be related to the guluronic acid content (“G content”) of the alginate as well as the arrangement of guluronic and mannuronic acids on the polymer chain. The G content of the alginate used to form the gels is at least about 35%, preferably about 40% to about 80%, and more preferably about 40% to about 70%.

Alginate derived from, for example, Lessonia trabeculata and from the stems of Laminaria hyperborea have the necessary G content and can be used to form these gels. The amount of divalent cation required to react stoichiometrically with these G-blocks can be calculated for each alginate type by considering that two guluronic acid units plus one calcium ion are required to create one ionic crosslink. The amount of calcium required for stoichiometric saturation of a 1% sodium alginate solution are given in the following table:

| Seaweed Source | % G | mM Ca |
|---|---|---|
| Laminaria hyperborea (stem) | 70 | 14-16 |
| Laminaria hyperborea (leaf) | 54% | 11-13 |
| Lessonia trabeculata | 68% | 13-15 |

Complete saturation (100% saturation) of the gelling sites occurs when the composition contains exactly 1 mM divalent cation per 2 mM L-guluronic acid units (alginate) or per 2 mM D-galacturonic acid units (pectic substance). For example, a 15 mM calcium ion solution is required to 100% saturate a 1% solution of sodium alginate extracted from the stems of Laminaria hyperborea, a 12 mM calcium solution is required to 100% saturate a 1% solution of sodium alginate extracted from the leaves of Laminaria hyperborea, and a 14 mM solution of calcium ions is required to 100% saturate a 1% solution of sodium alginate extracted from Lessonia trabeculata. Thus, when alginate is used as the gel-forming polymer, the gel-forming composition preferably comprises 0.2 to 0.9 mM of divalent cation, preferably calcium (+2) ion, per 2 mM of L-guluronic acid units present in the alginate.

In the examples, the amount of the calcium present in the system is expressed in terms of % saturation, defined as the amount of calcium ion present as a percentage of the amount of calcium ion required to react with all the gelling sites present in the alginate. It is not assumed that all of the calcium present is available to react with alginate or that ionic crosslinks are formed with each calcium ion even after extended storage. However, because gelling agents such as dicalcium phosphate dihydrate, tricalcium phosphate, and tricalcium citrate dissociate almost completely at the acidic pH's produced in the examples, the percent saturation closely approximates the percent of ionic crosslinking or, as used herein, saturation.

In one method of preparing the gel, the first step is preparation of an aqueous dispersion containing all the ingredients except the pH modifier. If desired, the gel-forming polymer, the added polymer, and the gelling agent may be premixed and added as a dry blend. Although these ingredients may be added in any order, in one method a mixture of the active substance and a surfactant is prepared first. Then a dry blend of the gel-forming polymer, the added polymer, and the gelling agent is added to this mixture. Water is then added to this mixture to form the aqueous dispersion. The dispersion is preferably stirred at high speed to disperse the active substance in the aqueous dispersion. Other components, such as preservatives and colorants, can be added at suitable stages depending on their dispersion and solubility properties.

Before the pH modifier is added to the aqueous dispersion, at least a portion of the divalent cation in the aqueous dispersion is not available to ionically link the gel-forming polymer. Consequently, the aqueous dispersion will not gel in the absence of a pH modifier. The aqueous dispersion does not have to be prepared immediately before use to prevent gelation in the process equipment. Thus, to prevent premature gel formation, which might adversely affect the manufacturing process, it is advantageous to add the pH modifier after the other ingredients have been added to the gel-forming aqueous dispersion.

When the pH modifier is added to the aqueous dispersion, the divalent cation is solubilized or otherwise becomes available to react with the gelling sites due to the decrease in pH brought about by the pH modifier. Preferably the pH modifier slowly lowers the pH of the gel-forming composition. This slow release of divalent cation provides a gel that is mechanically homogeneous.

It is common practice to fill an air freshener gel container in one position and then change the container to another position after filling. For example, boat shaped containers, which are widely used for air treatment gels, are typically filled when lying flat and are then turned upright for packaging and display. It is preferred that the gelation should be rapid enough that the gel develops enough rigidity within 60 minutes, preferably within 30 minutes, to retain its shape when turned for storage.

As gelation takes place, after addition of the pH modifier, the pH of the gel-forming composition decreases. It is preferred that the pH should not decrease more than 2 pH units during the first 15 minutes after addition of the pH modifier and not more than 3 pH units during the first hour after addition of the pH modifier. This slow pH decrease is achieved by using pH modifier that slowly ionizes or slowly forms acid and by including a buffer in the system. A preferred pH modifier is glucono-delta-lactone, but other pH modifiers that slowly lower the pH could also be used.

In a preferred embodiment, the pH modifier is added immediately before the solution is added to the containers. The pH modifier can be added as a powder or as a solution. A preferred solution is between 5 and 25% glucono-delta-lactone in water. Preferably the glucono-delta-lactone solution is used within 1 hour of preparation, most preferably within 30 minutes.

### INDUSTRIAL APPLICABILITY

The gels are strong gels with good color and moderate to good clarity. The gel-forming polymer and the other ingredients used in gel-formation have excellent compatibility with surfactants, fragrance oils, preservatives and colorants. In addition, when this process is used with a divalent cation as the gelling agent, it has environmental advantages versus other cold processes involving gelling of polymers with trivalent metal ions, for example those processes involving a crosslinking reaction between carboxymethyl cellulose and aluminum.

A heating step is not required for gel formation. Thus, compared to processes that require a heating step and a cooling step for gel formation, manufacturing of the gel compositions of this invention has lower energy costs and improved production efficiencies because these steps have been eliminated along with the delay before lids to are applied containers of gel. This delay is necessary to allow gels to cool sufficiently to avoid condensation in the containers. Because gel formation does not start until the pH modifier is added, it is easy to recover from plant stoppages. As described above, the pH modifier can be added immediately before the solution is added to the containers so the likelihood of gel-forming in the processing equipment is greatly reduced.

The gels are useful for the controlled release of fragrant air fresheners, air deodorizers, pesticides, pest repellents and the like such as in the home (e.g., in rooms, closets, garages, workshops, storage areas, cabinets, refrigerators, etc), office, vehicles (e.g., cars, trucks, vans, trains, aircraft, watercraft, etc.) and industrial and institutional settings, including public restrooms, warehouses, factories, restaurants and stores. They are particularly useful as air fresheners, especially as automotive air fresheners. Automotive air fresheners are subjected to temperature extremes, especially during the summer and winter months and, in some climates, between nighttime and daytime. Because these gels have excellent high temperature stability (up to 180° F., 82° C.) and excellent freeze/thaw stability, they are especially well suited for use as automotive air fresheners.

The advantageous properties of this invention can be observed by reference to the following examples, which illustrate but do not limit the invention.

### EXAMPLES

Glossary

| | |
|---|---|
| Arylpon | Surfactant, glycerol mono/dilaurate polyglycol |
| GML20 | ether (Hoechst AG) |
| AKUCELL ® | Food grade CMC, Brookfield viscosity of a 1% |
| AF 2785 | aqueous solution at 25° C. is 1,500 to 2,500 mPas, |
| | measured with spindle 3 at 30 rpm (Akzo |
| | Nobel, Amersfoort, The Netherlands) |
| AKUCELL ® | Food grade CMC, Brookfield viscosity of a 1% |
| AF 2805 | aqueous solution at 25° C. is 2,500 to 4,500 mPas |
| | (Akzo Nobel, Amersfoort, The Netherlands) |
| AKUCELL ® | Food grade CMC, Brookfield viscosity of a 1% |
| AF 2985 | aqueous solution at 25° C. is 5,000 to 8,000 mPas, |
| | measured with spindle 4 at 30 rpm (Akzo |
| | Nobel, Amersfoort, The Netherlands) |
| AKUCELL ® | High viscosity food grade CMC (Akzo Nobel, |
| AF 3285 | Amersfoort, The Netherlands) |
| AKUCELL ® | High viscosity food grade CMC (Akzo Nobel, |
| AF 3295 | Amersfoort, The Netherlands) |
| Citron Vert | Fragrance oil (Specim, Belgium) |
| E3425/2 | |
| CMC-7HF | Food grade CMC, Brookfield viscosity of a 1% |
| | aqueous solution at 25° C. is 1,500 to 3,000 mPas, |
| | measured with spindle 3 at 30 rpm (Hercules, |
| | AQUALON ® Division, Wilmington, Del, USA) |
| Colportage | Fragrance oil (Drom, Germany) |
| CREMOPHOR ® | Surfactant, PEG 35 castor oil (BASF, |
| EL | Ludwigshaften, Germany) |
| CREMOPHOR ® | Surfactant, PEG 40 hydrogenated caster oil |
| RH 40 | (BASF, Ludwigshaften, Germany) |
| DCP | Dicalcium phosphate dihydrate (CaHPO₄ · |
| | 2H₂O; CAS No. 7789-77-7) |
| GDL | Glucono delta lactone; gluconinc acid δ-lactone |
| Green Tea | Fragrance oil (Dragoco Gerberding, |
| Fragrance | Holzminden, Germany) |
| K100 | METHOCEL ® K100; food grade hydroxypropyl |
| | methyl cellulose (Dow, Midland, MI, USA) |
| Menthe green | Green dye (Robertet, France) |
| Yellow E102 | Tartrazine, C.I. acid yellow 23 (Fusgaard, |
| | Copenhagen, Denmark) |

### Sample Preparation and Test Methods

### Sample Preparation

Samples were prepared using the following standard procedure except where noted. Fragrance oil was mixed with surfactant in a beaker. (In some cases vegetable oil was substituted for fragrance oil.) The gel-forming polymer, the added polymer, and gelling agent were dry blended and then added to the fragrance oil/surfactant mix. The resulting composition was blended for 1 min using a propeller mixer at low speed. Water was then added, followed by preservative and color. Mixer speed was increased to ensure good mixing. After the mixture had been mixed for 10 min, the pH modifier was added and mixing continued for a further 3 min.

The resulting gel-forming composition was poured into preweighed containers. About 100 to 120 g of the gel-forming composition was poured into a pot, which was sealed immediately with a screw-on cap. For evaluation of shape stability and evaluation of stand-up time, about 100 g to about 120 g of the gel-forming composition was poured into a boat-shaped container, which was sealed with a flexible adhesive seal.

### Processing Viscosity and pH

All the ingredients of the gel-forming composition except the pH modifier were combined and mixed for 10 min. The viscosity of this composition was measured using a Brookfield viscometer Model RVD-II using appropriate spindles, typically spindle A, at 2.5 rpm. The viscosity reading was recorded after 15 seconds.

The pH of the composition was measured immediately before adding the pH modifier. Then the pH modifier was added and the pH was measured at various time intervals after the addition.

### Gel-Point Time

The gel-forming composition was poured into a pot and allowed to stand at room temperature. The open container was tilted sideways every 5 min for the first 20 min and thereafter every 15 min as required to observe whether the mixture remained liquid or had gelled. The gel-point time is the first observation time at which the contents no longer flowed freely.

### Stand-Up Time

Sealed boats were maintained in a horizontal position for a predetermined time period after filling and then were stood vertically after an initial gel set time had elapsed. Typically five boats were filled and one boat stood vertically after 10, 20, 30, 40, and 50 min, respectively. After storage in a vertical position for one day, the gels were evaluated visually. The stand-up time was recorded as the shortest time interval for which the gel remained in contact with all side surfaces of the boat.

### Shape Stability

Sealed boats were kept horizontally for 30 min after filling, then turned vertically. After storage in a vertical position at room temperature (approximately 20° C.) for 1 month, the shape retention of the gels was evaluated visually. Gels were rated as having ‘Good’ shape stability if the gel remained in contact with all side surfaces of the boats, and rated as ‘Bad’ if any gaps had developed between the gel and the side surfaces.

### Gel Strength

Sealed pots containing gels were placed in a 20° C. waterbath and removed after designated time periods. The break strength of the gel was immediately measured as the force (in grams) required to fracture the top surface of gels using a TA-XT2 Texture Analyzer fitted with an 11 mm diameter probe using a rate of descent of 1 mm/s.

### % Syneresis

All free moisture was removed from the gel and from inside of the gel container and lid using pre-weighed blotting paper. Syneresis was calculated by expressing the free moisture as a % of the original gel weight.

### Freeze/Thaw Storage Cycle

Eighteen hours after the gel preparation, sealed pots were placed in a freezer set at −15° C. and stored for 18 hours. The pots were transferred to a 20° C. waterbath for 2 hours prior to measurement of % syneresis.

### High Temperature Storage Stability

Eighteen hours after gel preparation, gel-containing sealed pots were placed in an oven at the indicated temperatures and stored for the indicated times. After they were removed from the oven, the samples were cooled in a 20° C. waterbath for 2 hours prior to measurement of % syneresis.

### Weight Loss During Use

Gels were removed from pots. Gels were weighed initially and after 28 days exposure to the atmosphere at room temperature (about 20° C.). The % weight loss was calculated.

In the following examples, which used sodium alginates extracted from Laminarea hyperborea (stem and leaf) and Lessonia trabeculata, having a viscosity (at 1 wt % alginate in water) of 183 cP, 170 cP and 150 cP, respectively, the stoichiometric saturation has been calculated using the molar amount of divalent cations in the mixture as a percentage of the level required for 100% saturation of the alginate using the mid-points of the ranges for alginate from a given seaweed source. Water hardness was taken into account in calculation of the total stoichiometric saturation. Tap water used in these examples contained 2 mM of calcium ions.

### Example 1

This example illustrates that gel formation does not take place in the absence of a pH modifier. Because gel formation does not occur in the absence of a pH modifier, there is a low risk of serious plant blockages if an interruption is experienced in production.

The stability of a solution of 1% alginate (Laminaria hyperborea leaf) and 0.344% calcium phosphate dihydrate was evaluated without addition of a pH modifier. The calcium ion was at 60% saturation of the alginate.

The dry ingredients were preblended and then dispersed in deionized water at high speed for 5 min. The solution was poured into gel dishes and stored at 20° C. overnight. The sample remained liquid. No gel was observed.

Compositions containing 1.5% alginate (L. trabeculata), 1% AKUCELL® AF 3285, 0.2% DCP, 2% Citron Vert fragrance oil and 5% CREMOPHOR® RH 40, were also evaluated without addition of a pH modifier.

The dry ingredients were preblended and then dispersed a mixture of fragrance oil and surfactant. In one example, deionized water was added to this mixture. In a second example, tap water was added. The solutions were stored overnight at room temperature (about 20° C.). Both samples remained liquid. No gelation was observed.

### Example 2

This Example shows the effect of different organic acids on gel stability. The pH modifiers that are known to reduce pH quickly (such as lactic acid and acetic acid) create gels that have higher syneresis than gels produced using a slower pH modifier (such as glucono-delta-lactone).

Gels were prepared using the gel-forming compositions shown in Table 1A. The pH modifier was added last and mixed in for 1 minute prior to pouring the mixtures into pots. The target pH was 5.0. The samples prepared using 10% aqueous solutions of lactic acid and of acetic acid started to gel immediately after the acid was added. The properties of the resulting gels are shown in Table 1B.

TABLE 1A^{a}

| | Example # | | | | | | |
|---|---|---|---|---|---|---|---|
| | 2-1 | 2-2 | 2-3 | 2-4 | 2-5 | 2-6 | 2-7 |
| Vegetable oil | 2.0 | 2.0 | 2.0 | 2.0 | 2.0 | 2.0 | 2.0 |
| Arylpon GML20 | 5.0 | 5.0 | 5.0 | 5.0 | 5.0 | 5.0 | 5.0 |
| Alginate^{b} | 1.5 | 1.5 | 1.5 | 1.5 | 1.5 | 1.5 | 1.5 |
| DCP | 0.33 | 0.44 | 0.33 | 0.33 | 0.33 | 0.33 | 0.33 |
| Guar gum | 0 | 0 | 0.5 | 0 | 0 | 0 | 0 |
| GDL | 0.4 | 0.4 | 0.4 | 0 | 0 | 0 | 0 |
| Lactic acid (10%) | 0 | 0 | 0 | 2.0 | 4.0 | 0 | 0 |
| Acetic acid (10%) | 0 | 0 | 0 | 0 | 0 | 2.0 | 4.0 |
| Tap water | 90.77 | 90.16 | 90.27 | 89.17 | 87.17 | 89.17 | 87.17 |

^{a}% wt
^{b}From Laminaria hyperborea leaf, viscosity 170 cP.

TABLE 1B

| | Example # | | | | | | |
|---|---|---|---|---|---|---|---|
| | 2-1 | 2-2 | 2-3 | 2-4 | 2-5 | 2-6 | 2-7 |
| gel strength | 832 | 801 | 761 | ND^{a} | ND | ND | ND |
| pH after | 4.94 | 4.99 | 4.89 | 5.07 | 4.26 | 4.74 | 4.35 |
| 18 hrs | | | | | | | |
| pH after | 4.97 | 5.03 | 4.87 | ND | ND | ND | ND |
| 70 hrs | | | | | | | |
| % syneresis | | | | | | | |
| 17 hrs @ | 0.50 | 0.35 | 0.52 | 1.83 | 6.94 | 1.0 | 5.0 |
| 20° C. | | | | | | | |
| freeze thaw | 11.97 | 2.3 | 5.75 | ND | ND | ND | ND |

^{a}Not Determined.

### Example 3

This example illustrates formation of gels with citric acid as pH modifier.

A gel-forming composition was prepared using the following formulation: Citron vert fragrance, 2.0%; CREMOPHOR® RH 40, 5.0%; alginate (Laminaria hyperborea stem), 1.5%; AKUCELL® AF 3285, 0.33%; DCP, 0.256%; 0.5% Yellow E102 in water, 0.25%; chloroacetamide, 0.1%; and tap water, 90.564%.

A citric acid solution was added to 150 g samples of this mixture. The citric acid solution was poured into boats or pots before, during, or after addition of the mixtures. Citric acid is an immediate pH modifier. The properties of the resulting gels are shown in Table 2.

TABLE 2
Gel Properties for Citric Acid Level and Addition Sequence

| | | | | homo- | |
|---|---|---|---|---|---|
| | pH modifier | method | pH | geneity | syneresis |
| 3-1 | 1 ml 20% citric acid | in boat before | 6.03 | some- | very low |
| | | | | what | |
| 3-2 | 1 mL 20% citric acid | in boat after | 3.54 | no | moderate |
| 3-3 | 1 mL 20% citric acid | in boat during | 5.06 | yes | very low |
| 3-4 | 2 mL 20% citric acid | in boat during | 4.11 | no | moderate |
| 3-5 | 4 mL 20% citric acid | in boat during | 3.33 | no | high |
| 3-6 | 10 mL 5% citric acid | in boat during | 3.64 | no | high |
| 3-7 | 5 mL 5% citric acid | in boat during | 3.74 | no | high |
| 3-8 | 2 mL 15% citric acid | in boat during | 3.64 | no | very low |
| 3-9 | 3 mL 15% citric acid | in pot during | 3.29 | no | verv low |

The homogeneity and syneresis of resulting gels were assessed after 15 days storage at room temperature. Homogeneity, assessed visually, was poor in most samples. The sample in which 1 mL of 20% the citric acid was added during the addition of the mixture to the container was visually homogeneous and showed no syneresis. However, it was a soft gel of insufficient strength. The higher pH of this sample resulted in lower availability of calcium resulting in a weak gel.

This example illustrates that a pH modifier that reduces pH rapidly does not provide good functionality. Regardless of the method of addition of citric acid, the resulting gels were formed too rapidly (due to the rapid release of calcium), were not homogeneous and/or resulted in excessive syneresis on storage.

### Example 4

This example illustrates the buffering effect of DCP.

The pH was measured as a function of time after addition of 0.4% GDL powder to tap water (4-1) and of 0.4% GDL powder to tap water containing 0.33% DCP (4-2). The results are shown in Table 3.

TABLE 3

| Time (min) | 4-1 | 4-2 |
|---|---|---|
| 1 | 5.4 | 6.1 |
| 2 | 4.6 | 5.9 |
| 3 | 4.1 | 5.76 |
| 30 | ND^{a} | 4.7 |
| 60 | 3.15 | ND |
| 240 | 2.85 | 4.2 |

^{a}ND = not determined

### Example 5

This example illustrates both the effect of carboxymethyl cellulose viscosity on gel stability and the effect of high saturation levels on storage stability. Gel-forming compositions containing different commercial grades of carboxymethyl cellulose were prepared.

Gels were prepared using the following composition: 2.0% vegetable oil; 5.0% Arylpon GML surfactant; 1.5% alginate (Laminaria hyperborea leaf, viscosity 170 cP); carboxymethyl cellulose as shown in Table 4A; 0.33% DCP; 0.40% GDL, and tap water to 100%. Syneresis was measured for each gel sample after 17 hours of storage at room temperature and after one freeze thaw cycle. The results are given in Table 4A and Table 4B.

TABLE 4A

| | Sample | | | | |
|---|---|---|---|---|---|
| | 5-1 | 5-2 | 5-3 | 5-4 | 5-5 |
| AKUCELL ® AF 2785 | 0 | 0.25 | 0.50 | 0 | 0 |
| AKUCELL ® AF 2805 | 0 | 0 | 0 | 0.25 | 0.50 |
| pH after 18 hours | 4.86 | 4.94 | 5.06 | 4.85 | 5.00 |
| % syneresis after 17 hr | 0.77 | 0.50 | 0.36 | 0.57 | 0.37 |
| % syneresis after freeze/thaw | 14.44 | 1.95 | 2.26 | 3.27 | 1.91 |

TABLE 4B

| | Sample | | | | | |
|---|---|---|---|---|---|---|
| | 5-6 | 5-7 | 5-8 | 5-9 | 5-10 | 5-11 |
| AKUCELL ® AF 2985 | 0.25 | 0.50 | 0 | 0 | 0 | 0 |
| AKUCELL ® AF 3285 | 0 | 0 | 0.25 | 0.50 | 0 | 0 |
| AKUCELL ® AF 3295 | 0 | 0 | 0 | 0 | 0.25 | 0.50 |
| pH after 18 hours | 4.92 | 4.98 | 4.85 | 4.97 | 4.97 | 4.92 |
| % syneresis after 17 hr | 0.31 | 0.39 | 0.46 | 0.38 | 0.46 | 0.29 |
| % syneresis after freeze/thaw | 6.80 | 2.00 | 2.11 | 0.75 | 8.57 | 0.77 |

The stoichiometric saturation was 144%. These gels show the superior freeze/thaw stability obtained by using a high viscosity CMC (AKUCELL® AF 3285 and AKUCELL® 3295) in the alginate gel composition.

Additional gels were prepared using various alginates at 1.5% and having the following compositions: 0.33% AKUCELL® AF 3285, 0.33% DCP, 0.4% GDL, 2% Colportage, 5% CREMOPHOR® EL, with the balance being tap water.

TABLE 4C

L. hyperborea

| Alginate | stem | leaf | L. trabeculata |
|---|---|---|---|
| Alginate viscosity | 183 cP | 170 cP | 150 cP |
| % saturation | 115 | 144 | 123 |
| % syneresis at 4 days | 0.98 | 1.40 | 1.37 |
| % syneresis at 12 days | 1.60 | ND | ND |

Saturation of greater than 100% resulted in excessive syneresis with storage times of greater than 17 hours.

### Example 6

This example illustrates the effect of the added polymer on freeze/thaw and heat stability. This example also illustrates that a high viscosity added polymer provides the best control of syneresis after storage under varying time and temperature test conditions. In addition, it illustrates the beneficial effect of saturation levels less than 100% on long term storage stability.

Air freshener gels (500 g batch size) were prepared with several different added polymers. The formulation used was: 3.2% vegetable oil; 8.0% CREMOPHOR® RH40; 1.60% alginate from Lessonia trabeculata; 0.26% dicalcium phosphate dihydrate; 0.8% E-102 (0.3% in water); 0.16% chloroacetamide; added polymer as indicated in Table 5; 0.42% GDL; and the balance tap water. The stoichiometric saturation was 82%.

TABLE 5
Stability of Gels Containing Different Added Polymers

| | Polymer | | | | | | | | | |
|---|---|---|---|---|---|---|---|---|---|---|
| | 6-1 | 6-2 | 6-3 | 6-4 | 6-5 | 6-6 | 6-7 | 6-8 | 6-9 | 6-10 |
| Alginate | 1.60 | 1.60 | 1.60 | 1.60 | 1.60 | 1.60 | 1.60 | 1.60 | 1.60 | 1.60 |
| Xanthan | 2.40 | 1.60 | 0 | 0 | 0 | 0 | 0 | 0 | 0 | 0 |
| K100 | 0 | 0 | 2.40 | 1.60 | 0 | 0 | 0 | 0 | 0 | 0 |
| AF 2785 | 0 | 0 | 0 | 0 | 1.60 | 0 | 0 | 0 | 0 | 0 |
| AF 2805 | 0 | 0 | 0 | 0 | 0 | 1.60 | 0 | 0 | 0 | 0 |
| AF 2985 | 0 | 0 | 0 | 0 | 0 | 0 | 1.60 | 0 | 0 | 0 |
| CMC-7HF | 0 | 0 | 0 | 0 | 0 | 0 | 0 | 1.60 | 0 | 0 |
| AF 3285 | 0 | 0 | 0 | 0 | 0 | 0 | 0 | 0 | 2.40 | 1.60 |
| Viscosity^{a} | 5424 | 3696 | 8240 | 3892 | 2800 | 2640 | 4048 | 3200 | 8528 | 3956 |
| Gel strength^{b} | 149 | 228 | 306 | 326 | 58 | 55 | 51 | 41 | 115 | 139 |
| pH @ 18 hr | 5.37 | 5.37 | 5.03 | 5.28 | 5.45 | 5.66 | 5.59 | 6.30 | 5.81 | 5.42 |

% syneresis @ 20° C.

| | | | | | | | | | | |
|---|---|---|---|---|---|---|---|---|---|---|
| 18 hr | 0.27 | 0.36 | 0.25 | 0.30 | 1.16 | 0.53 | 0.40 | 0.89 | 0.38 | 0.33 |
| 7 days | 0.22 | 0.32 | 0.57 | 0.65 | 0.49 | 0.40 | 0.65 | 0.50 | 0.32 | 0.41 |

% syneresis after

| | | | | | | | | | | |
|---|---|---|---|---|---|---|---|---|---|---|
| 1 f/t cycle | 0.31 | 0.44 | 0.49 | 1.24 | 1.03 | 1.02 | 1.38 | 1.40 | 0.63 | 0.62 |
| 6 hr @ 60° C. | 0.29 | 0.27 | 0.23 | 0.32 | 0.64 | 0.54 | 0.64 | 0.80 | 0.49 | 0.28 |
| 6 hr @ 80° C. | 1.55 | 1.04 | 6.73 | 0.41 | 3.34 | 2.87 | 2.53 | 0.72 | 3.91 | 0.91 |

^{a}in cP
^{b}in g.

### Example 7

This example illustrates the pH change as a function of time during gelation. There was a controlled pH decrease of no more than 2 pH units in the first 15 min and no more than 3 pH units in the first hour.

Gel samples with a stoichiometric saturation of 125% were prepared using an alginate from Lessonia trabeculata in the following formulation: fragrance oil, 2.0%; CREMOPHOR® EL, 5.0%; alginate, 1.5%; AKUCELL® AF 3285, 0.33%; DCP, 0.4%; tap water, 90.0275; 0.35% Menthe green in water, 0.3125%; chloroacetamide, 0.1%; and GDL, 0.4%.

The pH before GDL addition was 7.17. The pH at 5 min after GDL addition was 6.63. The pH at 15 minutes after addition was 6.36. The pH at 1 hour after addition was 5.74. The pH at 18 hours after addition was 5.12. The pH at 4 days after addition was 4.98. The pH at 5 days after addition was 4.98. The pH at 14 days after addition was 5.12.

The gel break strength at 18 hours at 20° C. was 943 grams. The syneresis at 17 hours at 20° C. was 0.44%. The syneresis after 1 freeze thaw cycle was 1.98%.

### Example 8

This example illustrates gels with different calcium ion sources.

Gel-forming compositions were prepared using the calcium ion sources indicated in Table 6 in the following formulation with the balance to 100% being tap water: 2.0% Green Tea Fragrance; 5.0% Arlypon GML 20; 1.5% Laminaria hyperborea stem alginate; 0.5% GDL; 0.33% AKUCELL® AF 3285; 0.25% Menthe green (0.3%) in water); and 0.1% chloroacetamide.

TABLE 6
Effect of Calcium Ion Source on Gel Properties

| | 8-1 | 8-2 | 8-3 | 8-4 |
|---|---|---|---|---|
| % saturation | 68% | 68% | 68% | 79% |
| dicalcium phosphate dihydrate | 0.21 | 0 | 0 | 0.25 |
| tricalcium phosphate | 0 | 0.17 | 0 | 0 |
| tricalcium citrate (4 H2O) | 0 | 0 | 0.24 | 0 |
| sodium hexametaphosphate | 0 | 0 | 0 | 0.10 |
| pH before adding GDL | 7.40 | 7.08 | 7.09 | 7.72 |
| pH at 15 minutes | 6.43 | 6.15 | 6.14 | 6.71 |
| pH at 1 hour | 6.09 | 5.82 | 5.57 | 6.21 |
| pH at 18 hour | 4.49 | 4.71 | 4.56 | 5.23 |
| pH at 2 days | 4.38 | 4.60 | 4.47 | 5.06 |
| pH at 7 days | 4.40 | 4.67 | 4.39 | 5.13 |
| pH at 14 days | 4.36 | 4.63 | 4.38 | 5.12 |
| gel-point time (minutes) | 15 | 90 | 20 | 75 |
| % syneresis at 17 hours | 0.36 | 0.28 | 0.36 | 0.45 |
| % syneresis in 7 days | 1.00 | 0.81 | 0.79 | 0.95 |
| % syneresis at 14 days | 1.30 | 1.07 | 1.11 | 1.29 |
| % syneresis, freeze thaw | 1.15 | 0.53 | 0.71 | 3.48 |
| break strength at 18 hours (g) | 841 | 521 | 657 | 1000 |

This example illustrates several ingredients that provide calcium ions and are suitable for use in the gels. Example 8-1 and 8-3 each illustrate a formulation that provides acceptable gel properties with reasonably fast gelation times of fifteen to twenty minutes. Although Example 8-2 had a significantly longer gelation time of ninety minutes, this formulation provided the lowest freeze/thaw syneresis of the four gels tested.

### Example 9

This example illustrates the effect of stoichiometric saturation.

The effect of calcium stoichiometry to the alginate was evaluated using the following formulation with the level of DCP as indicate in Table 7, the balance to 100% being tap water. The % saturation was calculated taking into account the hardness of the tap water. The gel-forming composition contained: 2.0% vegetable oil; 5.0% CREMOPHOR® EL; 1.5% Laminaria hyperborea stem alginate; 0.33% AKUCELL® AF 3285; 0.25% Menthe green (0.3%) in water); 0.1% chloroacetamide; and 0.31% GDL.

TABLE 7
Effect of Stoichiometric Saturation

| | 9-1 | 9-2 | 9-3 |
|---|---|---|---|
| % stoichiometric saturation | 98% | 79% | 68% |
| dicalcium phosphate dihydrate | 0.33% | 0.26% | 0.21% |
| pH at 15 minutes | 6.24 | 5.89 | 5.94 |
| pH at 1 hour | 5.48 | 5.41 | 5.37 |
| pH at 18 hour | 4.87 | 4.76 | 4.61 |
| pH at 4 days | 4.81 | 4.67 | 4.50 |
| pH at 7 days | 4.97 | 4.65 | 4.47 |
| gel-point time (minutes) | 90 | 90 | 90 |
| % syneresis at 17 hours | 0.36 | 0.39 | 0.33 |
| % syneresis at 12 days | 1.71 | 1.66 | 0.86 |
| % syneresis, freeze thaw | 0.69 | 0.55 | 0.59 |
| break strength at 18 hours (g) | 789 | 737 | 449.5 |

### Example 10

This example illustrates the effect of stoichiometric saturation with varying GDL levels.

The effect of calcium stoichiometry to the alginate was evaluated using the following gel-forming composition with the amount of DCP and GDL indicated in Table 8, with the balance being tap water. The % saturation was calculated taking into account the hardness of the tap water. The gel-forming composition contained: 2.0% Green Tea fragrance; 5.0% Arlypon GML 20; 1.5% Laminaria hyperborea stem alginate; 0.33% AKUCELL® AF 3285; 0.25% Menthe green (0.3%) in water; and 0.1% chloroacetamide.

TABLE 8
Gel Compositions with Varied Levels of GDL

| | 10-1 | 10-2 | 10-3 | 10-4 | 10-5 |
|---|---|---|---|---|---|
| % calcium saturation | 79% | 79% | 68% | 57% | 57% |
| DCP | 0.26% | 0.26% | 0.21% | 0.17% | 0.17% |
| GDL | 0.40% | 0.60% | 0.50% | 0.40% | 0.60% |
| pH before adding GDL | 7.40 | 7.42 | 7.40 | 7.46 | 7.38 |
| pH at 15 minutes | 6.53 | 6.63 | 6.43 | 6.70 | 6.62 |
| pH at 1 hour | 6.21 | 6.28 | 6.09 | 6.14 | 5.84 |
| pH at 18 hour | 4.98 | 4.45 | 4.49 | 4.59 | 4.27 |
| pH at 48 hours | 4.87 | 4.34 | 4.38 | 4.48 | 4.16 |
| pH at 7 days | 4.96 | 4.32 | 4.40 | 4.50 | 4.16 |
| pH at 14 days | 4.87 | 4.25 | 4.36 | 4.56 | 4.17 |
| gel-point time (minutes) | 15 | 15 | 15 | 20 | 20 |
| % syneresis at 17 hours | 0.39 | 0.42 | 0.36 | 0.33 | 0.33 |
| % syneresis at 7 days | 0.95 | 0.86 | 1.00 | 0.62 | 0.38 |
| % syneresis 14 days | 0.88 | 2.01 | 1.30 | 0.73 | 1.08 |
| % syneresis, freeze thaw | 1.30 | 3.63 | 1.15 | 0.54 | 0.91 |
| break strength at 18 hours (g) | 1117 | 1147.5 | 841 | 488 | 550 |

### Example 11

This example illustrates the effect of GDL level on the rate of gelation.

Gels were prepared using the following gel-forming composition with the amount of GDL as indicated in Table 9 with the balance being with tap water. The stoichiometric saturation was 81%. The gel-forming composition contained: 2.0% Green Tea fragrance; 5.0% Arlypon GML 20; 1.5% alginate from Lessonia trabeculata; 0.33% AKUCELL® 3285; and 1.00% DCP.

TABLE 9
Gels with Varying Levels of GDL

| | 11-1 | 11-2 | 11-3 | 11-4 |
|---|---|---|---|---|
| % GDL | 0.40 | 0.50 | 0.6 | 0.75 |
| standup time (min) | >50 | 50 | 40 | 30 |
| % syneresis in pot^{a} | 0.09 | 0.04 | 0.08 | 0.07 |
| % syneresis 16 hrs @ 52° C. | NT | NT | NT | 0.80 |

^{a}After 18 hours at 20° C.

### Example 12

This example illustrates the effect of deionized water (DI water) and tap water containing calcium ion.

Gels were prepared from Laminaria hyperborea stem alginate using the following gel-forming composition to compare performance between deionized water and tap water with a water hardness of 2 mM of calcium ion. The gel-forming compositions contained: 2.0% Citron Vert E3425/2; 5.0% CREMOPHOR® RH 40; 1.5% alginate; 0.33% AKUCELL® AF 3285; 0.17% DCP; 0.25% Menthe green (0.3% in water); 0.1% chloroacetamide; 90.15% water; and 0.5% GDL.

| | Tap Water | DI Water |
|---|---|---|
| % stoichiometric saturation | 57% | 44% |
| pH before adding GDL | 7.34 | 7.37 |
| pH at 15 min | 6.41 | 6.03 |
| pH at 1 hour | 5.81 | 5.65 |
| pH at 18 hour | 4.49 | 4.53 |
| pH at 8 days | 4.29 | 4.12 |
| % syneresis at 17 hours | 0.27 | 0.53 |
| % syneresis at 7 days | 0.41 | 0.58 |
| % syneresis, freeze thaw | 0.56 | 0.712 |
| gel break strength at 18 hr (g) | 460.5 | 223 |
| % loss at 28 days (open, RT) | 90.86 | 90.41 |

Having described the invention, we now claim the following and their equivalents.

1. A gel comprising:
(a) an active substance,
(b) a gel-forming polymer containing gelling sites, the gel-forming polymer selected from the group consisting of alginate, pectic substances, and combinations thereof, and
(c) at least one added polymer;
in which:
the gel-forming polymer is gelled;
the gelling sites of the gel-forming polymer are less that 100% saturated with a divalent cation or mixture of divalent cations; and
the at least one added polymer is not gelled by the divalent cation or mixture of divalent cations.

2. The gel of claim 1 in which the added polymer is carboxymethyl cellulose.

3. The gel of claim 1 in which the added polymer is a high viscosity carboxymethyl cellulose.

4. The gel of any of claims 1-3 in which the gel has a syneresis of <1% after 18 hours at 20° C. in a sealed container and <2% after one month storage at 20° C. in a sealed container.

5. The gel of any of claims 1-4 in which the pH of the gel is between 4 and 6.

6. The gel of any of claims 1-5 in which the gelled polymer is alginate.

7. The gel of any of claims 1-6 in which the alginate is 20% to 90% saturated.

8. The gel of any of claims 1-7 in which the alginate has a G content of at least 35%.

9. The gel of any of claims 1-8 in which the active substance comprises an ingredient selected from the group consisting of perfume constituents, pheromones, bactericides, insect attractants and repellants, animal attractants and repellants, insecticides, fungicides, and pharmaceutical and veterinary drugs.

10. The gel of any of claims 1-8 in which the active substance comprises a perfume constituent and a surfactant.

11. The gel of any of claims 1-10 in which the carboxymethyl cellulose has a Brookfield viscosity in 1% aqueous solution at 25° C. of between 10,000-20,000 cps, measured with a Brookfield viscometer using spindle 4 at 30 rpm.

12. The gel of any of claims 1-11 in which the divalent cation is calcium ion.

13. The gel of any of claims 1-12 in which the gel additionally comprises gluconic acid δ-lactone, the anion of gluconic acid δ-lactone, or a combination thereof.

14. The gel of any of claims 1 to 13 in which the gel is mechanically homogeneous.

15. A composition comprising about 45 to about 60 wt % of an alginate having a G content of about 40% to abut 90%, about 8 to about 20 wt % of carboxymethyl cellulose having a Brookfield viscosity in 1% aqueous solution at 25° C. of between 10,000-20,000 cps, and about 30 to 40 wt % of dicalcium phosphate.

16. The composition of claim 15 in which the amount of calcium ion present in the composition is less than the amount of calcium ion necessary to completely saturate the alginate present in the composition.

17. The composition of claim 15 in which the amount of calcium ion present in the composition is 40% to 90% of the amount of calcium ion necessary to completely saturate the alginate present in the composition.

18. The composition of any of claims 15 to 17 in which the alginate is isolated from Lessonia trabeculata.

19. A method for preparing a gel, the method comprising combining:
(a) an active substance,
(b) at least one gel-forming polymer containing gelling sites, the gel-forming polymer selected from the group consisting of alginate, pectic substances, and combinations thereof,
(c) at least one gelling agent comprising a divalent cation capable of gelling the gel-forming polymer,
(d) at least one added polymer,
(e) a pH modifier; and
(f) water;
in which:
the divalent cation is present in a molar amount less than that required to completely saturate the gelling sites of the gel-forming polymer, and
the added polymer is not gelled by the divalent cation.

20. The method of claim 19 in which:
the divalent cation is a divalent metal cation; and
the divalent cation is present in a molar amount of 20 to 90% of the amount required to completely saturate the gelling sites of the gel-forming polymer.

21. The method of claim 19 or claim 20 in which the pH modifier is added to a mixture of components (a), (b), (c), (d), and (f).

22. The method of any of claims 19-21 in which the gelling agent is calcium carbonate, dicalcium phosphate dihydrate, tricalcium phosphate, tricalcium citrate, or a mixture thereof.

23. The method of any of claims 19-22 in which the pH modifier is gluconic acid δ-lactone.

24. The method of any of claims 21-23 in which the pH of the mixture formed by addition of the pH modifier decreases by not more than 2 pH units during the first 15 minutes after addition of the pH modifier and not more than 3 pH units during the first hour after addition of the pH modifier.

25. The method of any of claims 18-24 in which:
the gelling agent is dicalcium phosphate dihydrate;
the pH of the resulting gel is between 4 and 6;
the gel-forming polymer is alginate;
the gelling sites are 20% to 90% saturated;
the alginate has a G content of at least 35%; and
the added polymer is a carboxymethyl cellulose having a Brookfield viscosity in 1% aqueous solution at 25° C. of between 10,000-20,000 cps, measured with a Brookfield viscometer using spindle 4 at 30 rpm.

26. A gel produced by the method of any of claims 19 to 25.

27. A gel-forming composition, the gel-forming composition comprising:
(a) an active substance,
(b) a gel-forming polymer containing gelling-sites, the gel-forming polymer selected from the group consisting of alginate, pectic substance and combinations thereof,
(c) a gelling agent comprising a divalent cation or mixture of divalent cations,
(d) at least one added polymer, and
(e) water
in which:
the amount of the divalent cation or mixture of divalent cations present is less than the amount required to completely saturate the gelling sites of the gel-forming polymer,
the gel-forming polymer is not gelled.

28. The gel-forming composition of claim 27 in which the gel additionally comprises gluconic acid δ-lactone, the anion of gluconic acid δ-lactone, or a combination thereof.

29. The gel-forming composition of claim 27 or 28 in which the gel-forming polymer is alginate having a G content of about 40% to about 90%; the added polymer is carboxymethyl cellulose having a Brookfield viscosity in 1% aqueous solution at 25° C. of between 10,000-20,000 cps, measured with a Brookfield viscometer using spindle 4 at 30 rpm, and the gelling agent is dicalcium phosphate or a hydrate thereof.

30. The gel-forming composition of any of claims 27 to 29 in which the gel-forming polymer is alginate, and the composition comprises 0.2 to 0.9 mM of divalent cation per 2 mM of L-guluronic acid units present in the alginate.

31. The gel-forming composition of any of claims 27 to 30 in which the divalent ion is calcium ion.
